(19) United States
(12) Patent Application Publication
(10) Pub. No.: US 2026/0248433 A1
(43) Pub. Date: Aug. 27, 2026

(54) BIOSIGNAL DETECTION DEVICE

(71) Applicant: SONY GROUP CORPORATION, TOKYO (JP)

(72) Inventors: RYO SASAKI, TOKYO (JP); MAO KATSUHARA, TOKYO (JP); KAZUNARI YOSHIFUJI, TOKYO (JP)

(21) Appl. No.: 18/714,346

(22) PCT Filed: Nov. 29, 2022
(86) PCT No.: PCT/JP2022/043852

§ 371 Date: May 29, 2024

(30) Foreign Application Priority Data
Dec. 7, 2021 (JP) 2021-198820
Aug. 8, 2022 (JP) 2022-126216

(51) Int. Cl. A61B 5/291; A61B 5/31; A61B 5/374

(52) U.S. Cl. CPC A61B 5/291; A61B 5/31; A61B 5/374

(57) ABSTRACT

A biosignal detection device according to the present disclosure includes a first electrode, a second electrode, a third electrode, and a first generator. The first electrode is configured to be in contact with a living body. The second electrode and the third electrode are configured to be in contact with the living body at respective positions different from that of the first electrode. The first generator generates a third signal related to the living body, on the basis of a first signal based on a potential of the first electrode and a second signal based on respective potentials of the second electrode and the third electrode.

### TECHNICAL FIELD

The present disclosure relates to a biosignal detection device.

### BACKGROUND ART

In existing brain wave measurements, a detection electrode is disposed at a position close to an active region of a brain, and a reference electrode is disposed in a part, such as earlobes, where propagation of brain waves is less. To observe brain waves with a high signal level, it is necessary to leave a sufficient distance between the reference electrode and the detection electrode, and to dispose the reference electrode at a limited position. This is a constraint in downsizing of an electroencephalograph. Meanwhile, in recent years, in terms of usability, an approach to acquire brain waves in a limited space, such as in or around an ear, has attracted attention, and there has been proposed a biosignal detection device that includes a reference electrode disposed in hollow of auricle and a detection electrode configured to be in contact with skin on a temporal bone and that measures brain waves using a potential difference between a potential of the reference electrode and a potential of the detection electrode (Patent Literature 1).

### CITATION LIST

### Patent Literature

PTL 1: Japanese Unexamined Patent Application Publication No. 2018-186934

### SUMMARY OF THE INVENTION

It is desired to improve detection performance of a biosignal detection device.

It is desirable to provide a biosignal detection device that makes it possible to improve the detection performance without limiting a position of a reference electrode.

A biosignal detection device as one embodiment of the present disclosure includes a first electrode, a second electrode, a third electrode, and a first generator. The first electrode is configured to be in contact with a living body. The second electrode and the third electrode are configured to be in contact with the living body at respective positions different from that of the first electrode. The first generator generates a third signal related to the living body, on the basis of a first signal based on a potential of the first electrode and a second signal based on respective potentials of the second electrode and the third electrode.

### BRIEF DESCRIPTION OF DRAWINGS

FIG. 1 is a diagram illustrating a configuration example of a biosignal detection device according to an embodiment of the present disclosure.

FIG. 2 is a diagram illustrating a configuration example of a sensor unit of the biosignal detection device according to the embodiment of the present disclosure.

FIG. 3 is a diagram illustrating a configuration example of reference electrodes of the biosignal detection device according to the embodiment of the present disclosure.

FIG. 4 is a diagram illustrating another configuration example of the reference electrodes of the biosignal detection device according to the embodiment of the present disclosure.

FIG. 5 is a diagram illustrating another configuration example of the reference electrodes of the biosignal detection device according to the embodiment of the present disclosure.

FIG. 6 is a diagram for describing an example of signal processing by the biosignal detection device according to the embodiment of the present disclosure.

FIG. 7 is a flowchart illustrating an operation example of the biosignal detection device according to the embodiment of the present disclosure.

FIG. 8 is a block diagram illustrating another configuration example of the sensor unit of the biosignal detection device according to the embodiment of the present disclosure.

FIG. 9 is a diagram describing another configuration example of the sensor unit of the biosignal detection device according to the embodiment of the present disclosure.

FIG. 10 is a diagram illustrating a configuration example of a sensor unit of a biosignal detection device according to Modification Example 1 of the present disclosure.

FIG. 11 is a diagram illustrating a configuration example of reference electrodes of the biosignal detection device according to Modification Example 1 of the present disclosure.

FIG. 12 is a diagram illustrating another configuration example of the reference electrodes of the biosignal detection device according to Modification Example 1 of the present disclosure.

FIG. 13 is a diagram illustrating another configuration example of the reference electrodes of the biosignal detection device according to Modification Example 1 of the present disclosure.

FIG. 14 is a diagram illustrating a configuration example of a sensor unit of a biosignal detection device according to Modification Example 2 of the present disclosure.

FIG. 15 is a diagram illustrating a configuration example of a sensor unit of a biosignal detection device according to Modification Example 3 of the present disclosure.

FIG. 16 is a diagram illustrating a configuration example of a sensor unit of a biosignal detection device according to Modification Example 4 of the present disclosure.

FIG. 17 is a diagram illustrating a configuration example of a sensor unit of a biosignal detection device according to Modification Example 5 of the present disclosure.

FIG. 18 is a diagram illustrating another configuration example of the sensor unit of the biosignal detection device according to Modification Example 5 of the present disclosure.

### MODES FOR CARRYING OUT THE INVENTION

In the following, an embodiment of the present disclosure will be described in detail with reference to the drawings. It is to be noted that the description will be given in the following order.
1. Embodiment
2. Modification Examples
2-1. Modification Example 1
2-2. Modification Example 2
2-3. Modification Example 3
2-4. Modification Example 4
2-5. Modification Example 5
2-6. Modification Example 6

### 1. EMBODIMENT

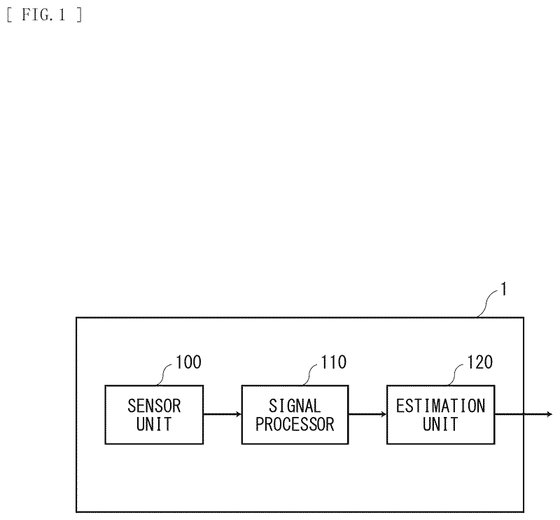

FIG. 1 is a diagram illustrating a configuration example of a biosignal detection device according to the embodiment of the present disclosure. A biosignal detection device 1 includes a sensor unit 100, a signal processor 110, and an estimation unit 120. The biosignal detection device 1 performs detection of a signal related to a living body (hereinafter referred to as a biosignal). It is possible to use the biosignal detection device 1 in an electronic apparatus that is wearable on a body, such as an ear, a head, a face, a neck, a hand, a wrist, an arm, a leg, or a chest.

The biosignal is, for example, a potential generated in association with activities of the living body. Specific examples include brain waves that are signals associated with brain activities, cardiac electrical activity that is a signal associated with heart activities, and muscular electrical activity associated with muscle activities. In the biosignal detection device 1, biosignals are obtained, making it possible to check a state of the living body.

The sensor unit 100 is a sensor configured to acquire a biosignal and outputs the biosignal, such as a signal related to brain waves, to the signal processor 110. The signal processor 110 and the estimation unit 120 include a processor, a memory, or the like, and perform signal processing (information processing) on the basis of a program. The signal processor 110 performs the signal processing such as frequency analysis processing, standardization processing, or the like, on the biosignal outputted from the sensor unit 100. The signal processor 110 outputs the biosignal after the signal processing to the estimation unit 120.

The estimation unit 120 uses the biosignal to perform processing of estimating the state of the living body. The estimation unit 120 performs, for example, processing of calculating a feature amount using the biosignal standardized by the signal processor 110. It is also possible to state that the estimation unit 120 analyzes the biosignal and extracts the feature amount. The feature amount is, for example, an α wave component, a β wave component, a γ wave component, or the like included in the biosignal. The estimation unit 120 estimates the state of the living body on the basis of a result of the calculation of the feature amount. As an example, the estimation unit 120 determines a psychological state such as whether or not the living body is relaxed, on the basis of the α wave component and the β wave component.

In addition, for example, the estimation unit 120 determines whether or not the living body is in a sleeping state, using the biosignal related to brain waves. As another example, the estimation unit 120 estimates a heart rate by analyzing the biosignal related to cardiac electrical activity. It is possible to state that the estimation unit 120 is a determination unit that determines the state of the living body. As such, analysis of the biosignal makes it possible to understand the state of the living body.

The estimation unit 120 may generate and output state information that is information indicating the state of the living body, as an estimation result. The state information includes, for example, information indicating whether or not the living body is in a relaxed state, information indicating whether or not the living body is in a sleeping state, information indicating a psychological state such as emotions of the living body, information indicating the heart rate, and the like. The state information may also be used to display an image representing the state of the living body or to output audio indicating the state of the living body.

At least one of the signal processor 110 or the estimation unit 120 or both of the signal processor 110 and the estimation unit 120 may be provided in an apparatus outside the biosignal detection device 1. Examples of an external apparatus include an electronic apparatus that is a terminal device (terminal) used by a user, a server, and the like. The electronic apparatus is a smart phone, a tablet terminal, a wearable terminal, a computer, or the like. It is also possible to collectively refer to the biosignal detection device 1 and the external apparatus as a biosignal detection device. Note that it is possible to collectively refer to the biosignal detection device 1 and the external apparatus that are coupled via a network as a biosignal detection device or a biosignal detection system.

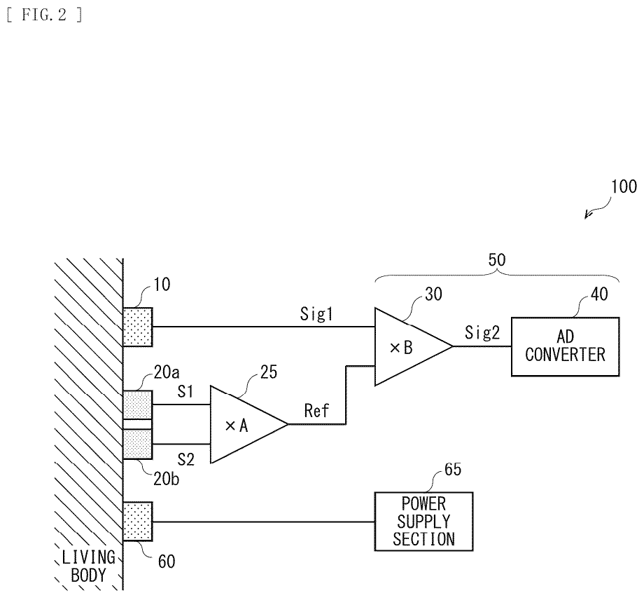

FIG. 2 is a diagram illustrating a configuration example of a sensor unit of the biosignal detection device according to the embodiment of the present disclosure. The sensor unit 100 is a sensor unit that includes a plurality of electrodes adapted to detect potentials and is configured to measure a biopotential. The sensor unit 100 includes an AFE (Analog Front End) section 50; an electrode (referred to as a measurement electrode 10) electrically coupled to the AFE section 50; a reference signal generator 25; and a plurality of electrodes (a reference electrode 20a and a reference electrode 20b in FIG. 2) electrically coupled to the reference signal generator 25.

The measurement electrode 10 and the reference electrodes 20a and 20b are each an electrode that includes an electrically conductive material and is configured to be in contact with the living body. The measurement electrode 10 and the reference electrodes 20a and 20b include, for example, aluminum (Al), copper (Cu), gold (Au), silver-silver chloride (Ag/AgCl), or the like. The measurement electrode 10 and the reference electrodes 20a and 20b may include an electrically conductive and elastic material.

The measurement electrode 10 and the reference electrodes 20a and 20b are spaced apart and are to be in contact with mutually different positions. The measurement electrode 10 is configured to be disposed at any position targeted for acquisition of a biosignal. In addition, the reference electrodes 20a and 20b are configured to be disposed at any respective positions including the vicinity of the measurement electrode 10. As an example, the reference electrode 20a and the reference electrode 20b may be disposed at a spacing (distance) of 30 mm to 40 mm or less that is considered a spatial resolution of brain waves.

In addition, in a case where a spacing between the measurement electrode 10 and the reference electrode 20a (or the reference electrode 20b) is D1 and a spacing between the reference electrode 20a and the reference electrode 20b is D2, the measurement electrode 10 and the reference electrodes 20a and 20b may be so disposed as to satisfy D1>>D2 and D2<40 mm. Alternatively, the measurement electrode 10 and the reference electrodes 20a and 20b may be so disposed as to satisfy D1>>D2 and D2<30 mm. The sensor unit 100 detects a potential (voltage) on a surface of the living body with the use of the measurement electrode 10 and the reference electrodes 20a and 20b. Due to electricity generated in the living body, a potential difference occurs between electrodes of the sensor unit 100 that are in contact with skin of the living body.

When actually used, the measurement electrode 10 comes into contact with a measurement site (measured part) and is given a potential of the contact site. The measurement electrode 10 is disposed, for example, directly above an activity region of a living body targeted for acquisition of a biosignal. The measurement electrode 10 is coupled to the AFE section 50 and supplies the AFE section 50 with a measurement signal Sig1. The measurement signal Sig1 is a signal corresponding to the potential of the site of the living body with which the measurement electrode 10 is in contact. The measurement signal Sig1 is a biosignal obtained by the measurement electrode 10.

The reference electrodes 20a and 20b each come into contact with the living body at a position different from that of the measurement electrode 10, and are each given a potential of a part in contact. The reference electrodes 20a and 20b may be disposed, for example, at any respective positions around the measurement electrode 10. The reference electrode 20a is coupled to the reference signal generator 25 and supplies the reference signal generator 25 with a signal S1 corresponding to the potential of the site of the living body with which the reference electrode 20a is in contact. In addition, the reference electrode 20b is coupled to the reference signal generator 25 and supplies the reference signal generator 25 with a signal S2 corresponding to the potential of the site of the living body with which the reference electrode 20b is in contact. The signal S1 is a biosignal obtained by the reference electrode 20a, and the signal S2 is a biosignal obtained by the reference electrode 20b.

In addition, in an example illustrated in FIG. 2, the sensor unit 100 includes a power supply section 65 and an electrode (referred to as a bias electrode 60) electrically coupled to the power supply section 65. The power supply section 65 includes a battery (storage battery), a converter, and the like, and is used to cause the sensor unit 100 to operate. The power supply section 65 supplies electric power to each section of the sensor unit 100. The bias electrode 60 is an electrode that includes an electrically conductive material and is configured to be in contact with the living body. The bias electrode 60 also includes, for example, aluminum (Al), copper (Cu), gold (Au), silver-silver chloride (Ag/AgCl), or the like. The bias electrode 60 may include an electrically conductive and elastic material.

The bias electrode 60 is electrically coupled to the living body and the power supply section 65, and is given a reference potential. The bias electrode 60 is an electrode for the reference potential. The bias electrode 60 that serves as the reference potential is electrically coupled to each section of the sensor unit 100. A potential of the bias electrode 60 serves as the reference potential (a ground potential, for example) for the measurement signal Sig1, the signal S1, the signal S2, or the like. It is also possible to state that the bias electrode 60 is an electrode adapted to define relative potentials of the sensor unit 100 and the living body.

The reference signal generator 25 generates a reference signal Ref on the basis of a plurality of signals obtained by a plurality of reference electrodes. In the present embodiment, the reference signal generator 25 generates the reference signal Ref based on a difference between the plurality of signals inputted by the plurality of reference electrodes and outputs the reference signal Ref to the AFE section 50. The reference signal generator 25 includes, for example, an amplifier circuit and may generate the reference signal Ref corresponding to a potential difference between the plurality of signals from the plurality of reference electrodes. The reference signal Ref is a signal defined by potentials of respective portions with which the plurality of reference electrodes is in contact. The reference signal Ref becomes a reference signal indicating a reference level for the measurement signal Sig1.

In the example illustrated in FIG. 2, to the reference signal generator 25, the reference electrode 20a inputs the signal S1 and the reference electrode 20b inputs the signal S2. The reference signal generator 25 generates the reference signal Ref based on a difference between the signal S1 and the signal S2. Using the potential of the bias electrode 60 as the reference potential, the reference signal generator 25 may generate the reference signal Ref based on a difference between a potential of the signal S1 and a potential of the signal S2. For example, the reference signal generator 25 includes a differential amplifier circuit and amplifies the difference between the signal S1 from the reference electrode 20a and the signal S2 from the reference electrode 20b by a predetermined gain (amplification rate) A. The gain A is so defined that a difference between the measurement signal Sig1 and the reference signal Ref is equal to or larger than a predetermined value.

As an example, the gain A is so set that a signal level of the reference signal Ref is smaller than the signal level of the measurement signal Sig1. The gain A may be so adjusted that an RMS value of the measurement signal Sig1 is sufficiently larger than the RMS value of the reference signal Ref. It is to be noted that the gain A is not limited to a value larger than 1 and may take a value smaller than or equal to 1. The reference signal generator 25 may output, to the AFE section 50, the reference signal Ref corresponding to the difference between the potential of the signal S1 and the potential of the signal S2.

FIG. 3 is a diagram illustrating a configuration example of reference electrodes of the biosignal detection device according to the embodiment of the present disclosure. The reference electrode 20a and the reference electrode 20b may have respective concentric shapes. As illustrated in FIG. 3, the reference electrode 20a may be provided concentrically on a perimeter of the reference electrode 20b. In the example illustrated in FIG. 3, the reference electrode 20a is provided at a spacing r from the reference electrode 20b. By doing so, it is possible to obtain the reference signal Ref with less noise, with the signal S1 of the reference electrode 20a and the signal S2 of the reference electrode 20b. This makes it possible to improve detection accuracy of a biosignal.

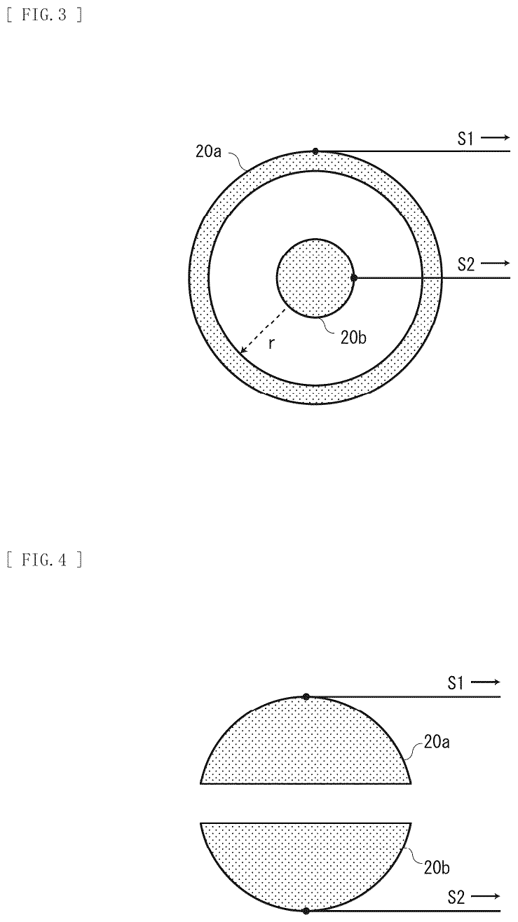

It is to be noted that shapes of the reference electrode 20a and the reference electrode 20b are appropriately modifiable and may each be circular, oval, or another shape. For example, the shapes of the reference electrode 20a and the reference electrode 20b may be configured to be circular as a whole, as illustrated in FIG. 4. In the example illustrated in FIG. 4, it is possible to state that the reference electrode 20a and the reference electrode 20b have respective shapes into which a circular electrode is divided.

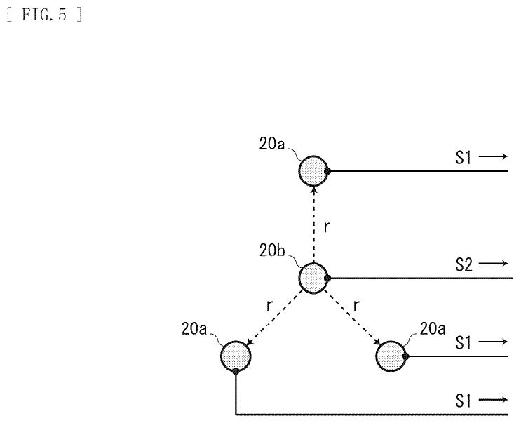

Each of the reference electrode 20a and the reference electrode 20b may include a plurality of electrodes. For example, as illustrated in FIG. 5, a plurality of reference electrodes 20a may be disposed around the reference electrode 20b. In this case, as in the example illustrated in FIG. 5, a spacing between the reference electrode 20b and each of the plurality of reference electrodes 20a may be an equal spacing r. It is to be noted that a shape of each of the measurement electrode 10 and the bias electrode 60 is also not specifically limited and may be circular, oval, or another shape. In addition, the shapes and the numbers of the measurement electrode 10, the reference electrodes 20a and 20b, and the bias electrode 60 are not limited to those in the illustrated examples.

As illustrated in FIG. 2, the AFE section 50 of the sensor unit 100 includes a biosignal generator 30 and an AD converter 40. The biosignal generator 30 generates a biosignal Sig2 on the basis of a measurement signal obtained by the measurement electrode 10 and a reference signal obtained by the reference signal generator 25. It is possible to state that the biosignal generator 30 is a signal detector that detects the biosignal Sig2. In the present embodiment, the biosignal generator 30 generates the biosignal Sig2 based on a difference between the measurement signal and the reference signal and outputs the biosignal Sig2 to the AD converter 40. The biosignal generator 30 includes, for example, an amplifier circuit and may generate the biosignal Sig2 corresponding to a potential difference between the measurement signal and the reference signal.

In the example illustrated in FIG. 2, to the biosignal generator 30, the measurement electrode 10 inputs the measurement signal Sig1 and the reference signal generator 25 inputs the reference signal Ref. The biosignal generator 30 generates the biosignal Sig2 based on the difference between the measurement signal Sig1 and the reference signal Ref. Using the potential of the bias electrode 60 as the reference potential, the biosignal generator 30 may generate the biosignal Sig2 based on a difference between a potential of the measurement signal Sig1 and a potential of the reference signal Ref. For example, the biosignal generator 30 includes a differential amplifier circuit and amplifies the difference between the measurement signal Sig1 from the measurement electrode 10 and the reference signal Ref from the reference signal generator 25 by a predetermined gain B. It is to be noted that the gain B is not limited to a value larger than 1 and may take a value smaller than or equal to 1. The biosignal generator 30 may output the biosignal Sig2 corresponding to the difference between the potential of the measurement signal Sig1 and the potential of the reference signal Ref to the AD converter 40.

As described above, the reference signal Ref is a signal corresponding to the difference between the respective potentials of the plurality of reference electrodes. In the present embodiment, the reference signal Ref that is a potential differential signal is used as the reference signal of the measurement signal Sig1. Therefore, it is possible to increase the difference between the reference signal Ref and the measurement signal Sig1. Even in a case where a contact position of the reference electrode and a contact position of the measurement electrode are close to each other, it is possible to ensure the difference between the reference signal Ref and the measurement signal Sig1, which allows the biosignal Sig2 to be detected with high accuracy. Calculation of the reference signal Ref with the signal difference between the plurality of reference electrodes reduces noise components propagated from a periphery of the electrodes, thus making it possible to obtain the biosignal Sig2 with, for example, brain wave components emphasized.

The AD converter 40 is an ADC (Analog to Digital Converter) and performs AD conversion processing on the biosignal Sig2 that is an analog signal outputted from the biosignal generator 30. The AD converter 40 outputs the biosignal Sig2 converted into a digital signal to the signal processor 110 illustrated in FIG. 1. The biosignal Sig2 outputted to the signal processor 110 is subjected to signal processing such as the frequency analysis processing by the signal processor 110, and thereafter outputted to the estimation unit 120. Next, a description will be given of an example of the signal processing by the signal processor 110.

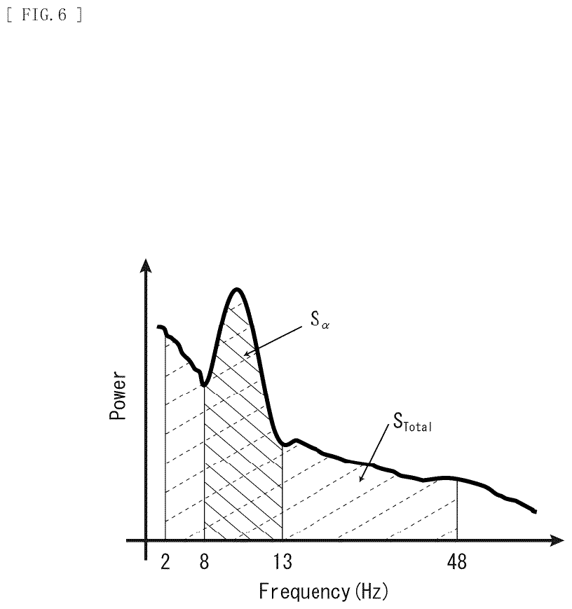

FIG. 6 is a diagram for describing an example of signal processing by the biosignal detection device according to the embodiment of the present disclosure. As an example, a description will be given of a case of the biosignal Sig2 indicating brain waves. In FIG. 6, a horizontal axis represents a frequency and a vertical axis represents signal intensity (Power). FIG. 6 illustrates characteristics of the biosignal Sig2 in terms of signal intensity (component) for each frequency.

The signal processor 110 calculates the signal intensity for each frequency by performing a frequency analysis on the biosignal Sig2. For example, as illustrated in FIG. 6, the signal processor 110 acquires a power distribution by performing fast Fourier transform processing on the biosignal Sig2. Brain waves are segmented to, for example, δ waves of 2 Hz to 4 Hz, θ waves of 4 Hz to 8 Hz, α waves of 8 Hz to 13 Hz, β waves of 13 Hz to 30 Hz, and γ waves of 30 Hz or greater. The signal intensity of each frequency band has a value corresponding to a brain activity state.

The signal processor 110 calculates standardized intensity (Relative power) as an index for comparing the signal intensity of each frequency band. The standardized intensity represents a ratio between total integrated intensity and integrated intensity of a specific wavelength band. The standardized intensity is normalized signal intensity. For example, for a waves, it is possible to express the standardized intensity with the following expression (1), by using the ratio of the integrated intensity S_{α} of 8 Hz to 13 Hz to the total integrated intensity S_{Total} of 2 Hz to 48 Hz, as illustrated in FIG. 6.

$$Relative power ( α ) = {S}_{α} / {S}_{Total} \qquad ( 1 )$$

In a manner similar to that in the case of the standardized intensity of a waves, the signal processor 110 may calculate the standardized intensity of other wavelength bands including, without limitation, β waves and γ waves. The signal processor 110 outputs a signal indicating the calculated standardized intensity to the estimation unit 120, as a biosignal after signal processing. For example, the estimation unit 120 extracts, as the feature amount, the standardized intensity of α waves from the biosignal after the signal processing, and estimates whether or not the body and mind are in a relaxed state, on the basis of the standardized intensity of α waves. In this manner, it becomes possible for the biosignal detection device 1 to analyze the biosignal Sig2, thereby checking the state of the living body, such as whether or not the relaxed state is achieved, or the like. Use of the biosignal on which the frequency analysis processing and the standardization processing have been performed makes it possible to capture characteristic changes that reflect activities of the living body (brain activities, for example).

It is to be noted that the standardization processing is not limited to the processing using the integrated intensity described above. An arithmetic expression other than the expression (1) described above may be used. It is to be noted that the signal processor 110 may output, as the biosignal after the signal processing, signal intensity obtained for each frequency through the frequency analysis to the estimation unit 120, without performing the standardization processing. For example, in a case where the sufficient signal intensity that is extractable as the feature amount is obtained, the signal processor 110 does not have to perform the standardization processing.

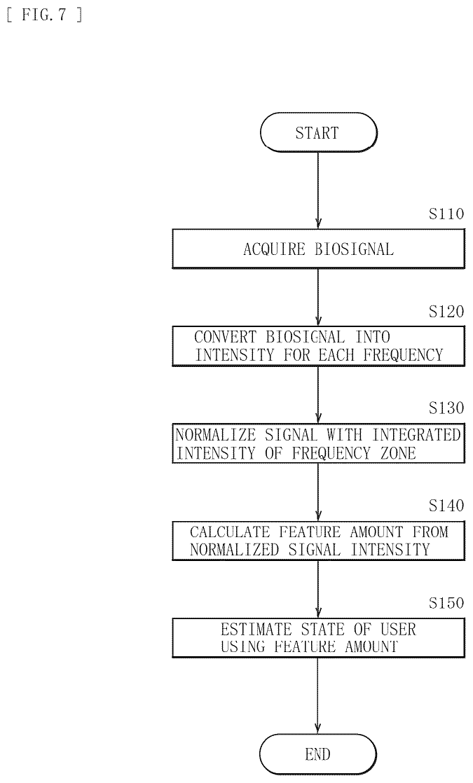

FIG. 7 is a flowchart illustrating an operation example of the biosignal detection device according to the embodiment of the present disclosure. A description will be given of an operation example of the biosignal detection device 1 with reference to the flowchart of FIG. 7. Processing illustrated in FIG. 7 is performed on the basis of a program stored in a memory, for example.

In step S110, the sensor unit 100 of the biosignal detection device 1 starts to measure a state of the user and acquires the biosignal Sig2 with the use of the measurement electrode 10 and the reference electrodes 20a and 20b. The sensor unit 100 performs the AD conversion processing and outputs, to the signal processor 110, the biosignal Sig2 that is the digital signal.

In step S120, the signal processor 110 performs the frequency analysis processing on the biosignal Sig2 to convert the biosignal Sig2 into the signal intensity for each frequency. In step S130, the signal processor 110 normalizes the converted signal intensity with the integrated intensity of a predetermined frequency zone. The signal processor 110 outputs, to the estimation unit 120, a signal indicating the normalized signal intensity as the biosignal after the signal processing.

In step S140, the estimation unit 120 calculates the feature amount from the signal intensity normalized by the signal processor 110. In step S150, the estimation unit 120 estimates the state of the user using the calculated feature amount. Thereafter, the biosignal detection device 1 ends the processing illustrated in the flowchart of FIG. 7.

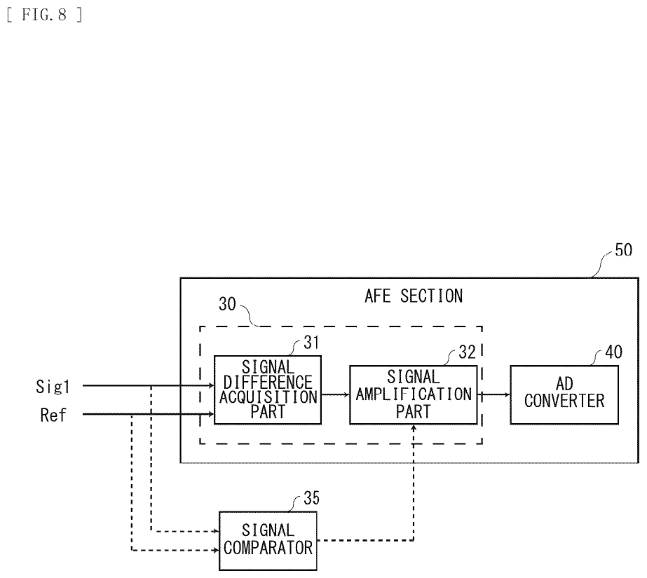

FIG. 8 is a block diagram illustrating another configuration example of the sensor unit of the biosignal detection device according to the embodiment of the present disclosure. In the example illustrated in FIG. 8, the sensor unit 100 includes a signal comparator 35. In addition, the biosignal generator 30 of the AFE section 50 includes a signal difference acquisition part 31 and a signal amplification part 32. The signal comparator 35 includes a comparator circuit, for example. It is to be noted that the signal difference acquisition part 31 and the signal amplification part 32 may be integrally configured.

To the signal comparator 35, the measurement electrode 10 (see FIG. 2) inputs the measurement signal Sig1 and the reference signal generator 25 inputs the reference signal Ref. The signal comparator 35 compares the measurement signal Sig1 and the reference signal Ref with each other and outputs an output signal that is a comparison result to the signal amplification part 32 of the AFE section 50. It is possible to state that the signal comparator 35 is a signal determination part and determines a magnitude relationship between the measurement signal Sig1 and the reference signal Ref. An output signal of the signal comparator 35 is a signal indicating the magnitude relationship between the measurement signal Sig1 and the reference signal Ref.

The signal difference acquisition part 31 outputs, to the signal amplification part 32, an output signal corresponding to the difference between the measurement signal Sig1 and the reference signal Ref. The signal amplification part 32 may amplify the output signal of the signal difference acquisition part 31 by the gain B and output the amplified signal as the biosignal Sig2. The signal amplification part 32 changes the gain B on the basis of the signal outputted from the signal comparator 35, that is, the output signal indicating the comparison result between the measurement signal Sig1 and the reference signal Ref. It becomes possible to adjust the gain B in accordance with the signal levels of the measurement signal Sig1 and the reference signal Ref. It is possible to state that the signal comparator 35 is a controller that controls the gain B. It is possible to so set the gain B that the signal level (signal amount) of the biosignal Sig2 is equal to or larger than a predetermined value and to ensure the signal level of the biosignal Sig2.

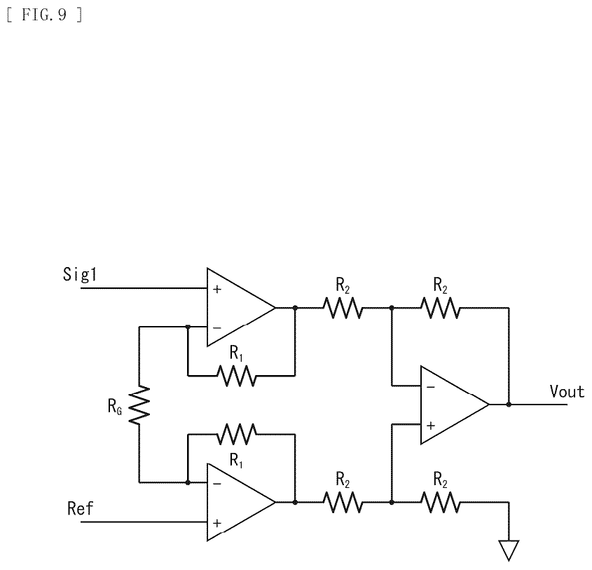

The signal amplification part 32 includes an instrumentation amplifier as illustrated in FIG. 9, for example. In the example illustrated in FIG. 9, it is possible to express an output signal Vₒᵤₜ of the signal amplification part 32 with the following expression (2):

$${V}_{out} = ( 1 + 2 {R}_{1} / {R}_{G} ) \times ( Sig 1 - Ref ) . \qquad ( 2 )$$

In the example illustrated in FIG. 9, a resistance value of a resistor RG being adjusted in accordance with the output signal of the signal comparator 35 makes it possible to change the gain B of the signal amplification part 32. It is possible for the signal amplification part 32 to output, as the biosignal Sig2, the output signal Vₒᵤₜ amplified by the gain B that is set on the basis of the comparison result obtained by the signal comparator 35.

It is to be noted that the signal comparator 35 may change the gain A of the reference signal generator 25 (see FIG. 2) described above, on the basis of the signal levels of the measurement signal Sig1 and the reference signal Ref. It is possible to state that the signal comparator 35 is a controller that controls the gain A. The reference signal generator 25 may output the reference signal Ref amplified by the gain A that is set on the basis of the comparison result obtained by the signal comparator 35.

### Workings and Effects

The biosignal detection device 1 according to the present embodiment includes a first electrode (the measurement electrode 10) that is configured to be in contact with a living body; a second electrode and a third electrode (the reference electrode 20a and the reference electrode 20b) that are configured to be in contact with the living body at respective positions different from that of the first electrode; and a first generator (the biosignal generator 30) that generates a third signal (the biosignal Sig2) related to the living body, on the basis of a first signal (the measurement signal Sig1) based on a potential of the first electrode and a second signal (the reference signal Ref) based on a potential of each of the second electrode and the third electrode.

The biosignal detection device 1 according to the present embodiment generates the reference signal Ref based on the difference between the respective potentials of the plurality of reference electrodes and uses the reference signal Ref as the reference signal of the measurement signal Sig1. Therefore, it is possible to increase the difference between the measurement signal Sig1 and the reference signal Ref. This makes it possible to detect a biosignal with high accuracy even in a case where the position of the reference electrode and the position of the measurement electrode are close to each other, and to improve detection performance of the biosignal.

In the present embodiment, detecting a biosignal using the reference signal Ref that is a potential differential signal makes it possible to observe, for example, significant brain waves that reflect changes in brain activities between the reference electrode and the measurement electrode that are close to each other. In addition, it becomes possible to suppress occurrence of constraints in the device shape.

Next, a description of modification examples of the present disclosure will be given. In the following, components similar to those of the above-described embodiment are denoted by the same reference numerals, and descriptions thereof are omitted where appropriate.

### 2. MODIFICATION EXAMPLES

### 2-1. Modification Example 1

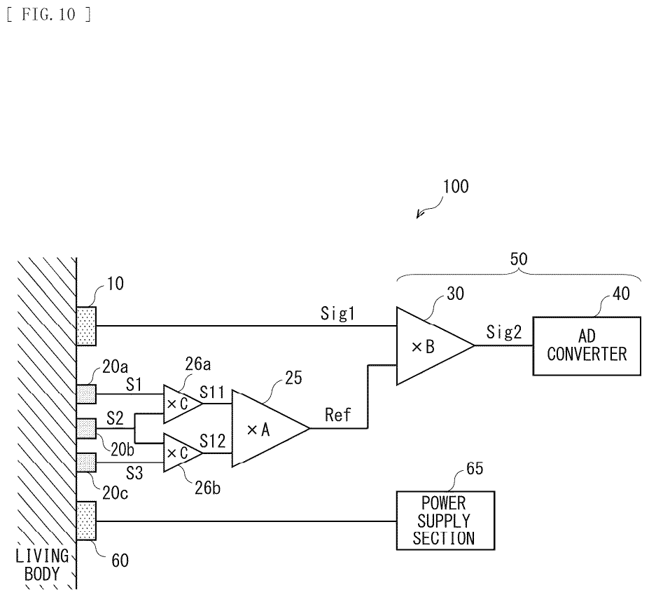

In the embodiment described above, the example is described in which the sensor unit 100 includes the two reference electrodes, but the number and the arrangement of the reference electrodes are not limited to this. The sensor unit 100 may include three or more reference electrodes. FIG. 10 is a diagram illustrating a configuration example of a sensor unit of a biosignal detection device according to Modification Example 1 of the present disclosure. The sensor unit 100 according to the present modification example includes signal generators 26a and 26b and reference electrodes 20a to 20c. The signal generator 26a is electrically coupled to the reference electrode 20a and the reference electrode 20b. In addition, the signal generator 26b is electrically coupled to the reference electrode 20b and the reference electrode 20c.

To the signal generator 26a, the reference electrode 20a inputs the signal S1 and the reference electrode 20b inputs the signal S2. The signal generator 26a includes, for example, a differential amplifier circuit, and generates a signal S11 obtained by amplifying the difference between the signal S1 and the signal S2 by a predetermined gain C. The signal generator 26a outputs, to the reference signal generator 25, the signal S11 corresponding to the difference between the potential of the signal S1 and the potential of the signal S2.

To the signal generator 26b, the reference electrode 20b inputs the signal S2 and the reference electrode 20c inputs a signal S3. The signal generator 26b includes, for example, a differential amplifier circuit and generates a signal S12 obtained by amplifying a difference between the signal S2 and the signal S3 by the gain C. The signal generator 26b outputs, to the reference signal generator 25, the signal S12 corresponding to a difference between a potential of the signal S2 and a potential of the signal S3.

The reference signal generator 25 generates a reference signal Ref obtained by amplifying a difference between the signal S11 outputted from the signal generator 26a and the signal S12 outputted from the signal generator 26b by the gain A. The reference signal generator 25 outputs, to the AFE section 50, the reference signal Ref corresponding to a difference between a potential of the signal S11 and a potential of the signal S12. In this manner, in the present modification example, it is possible to acquire the reference signal Ref based on the respective potentials of the three reference electrodes 20a to 20c. Use of the reference signal Ref makes it possible to detect the biosignal with high accuracy even in the case where the position of the reference electrode and the position of the measurement electrode are close to each other.

FIG. 11 is a diagram illustrating a configuration example of the reference electrodes of the biosignal detection device according to Modification Example 1. The reference electrodes 20a to the reference electrode 20c may have respective concentric shapes. In the example illustrated in FIG. 11, the reference electrode 20a is provided concentrically on the perimeter of the reference electrode 20b. The reference electrode 20a is provided at the spacing r from the reference electrode 20b. In addition, the reference electrode 20c is provided concentrically on the perimeter of the reference electrode 20a. The reference electrode 20c is provided at a spacing 2r from the reference electrode 20b.

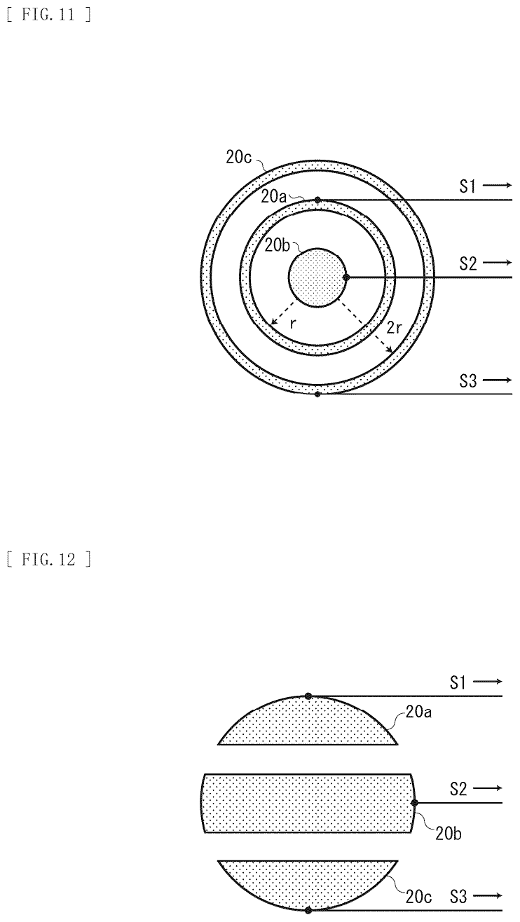

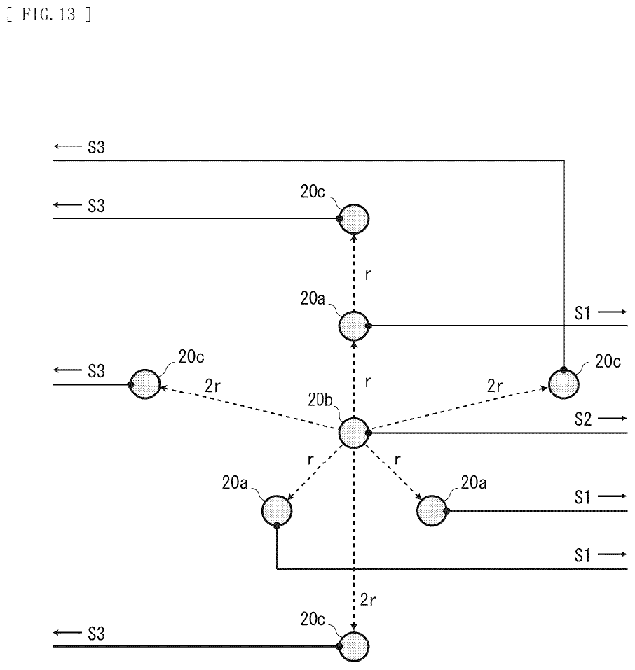

It is to be noted that shapes of the reference electrodes 20a to 20c are appropriately modifiable and may be configured to be circular as a whole, as illustrated in FIG. 12, for example. In the example illustrated in FIG. 12, it is possible to state that the reference electrodes 20a to 20c have respective shapes into which the circular electrode is divided. The reference electrode 20b is provided between the reference electrode 20a and the reference electrode 20c. The reference electrode 20a and the reference electrode 20c are disposed side by side with the reference electrode 20b in between.

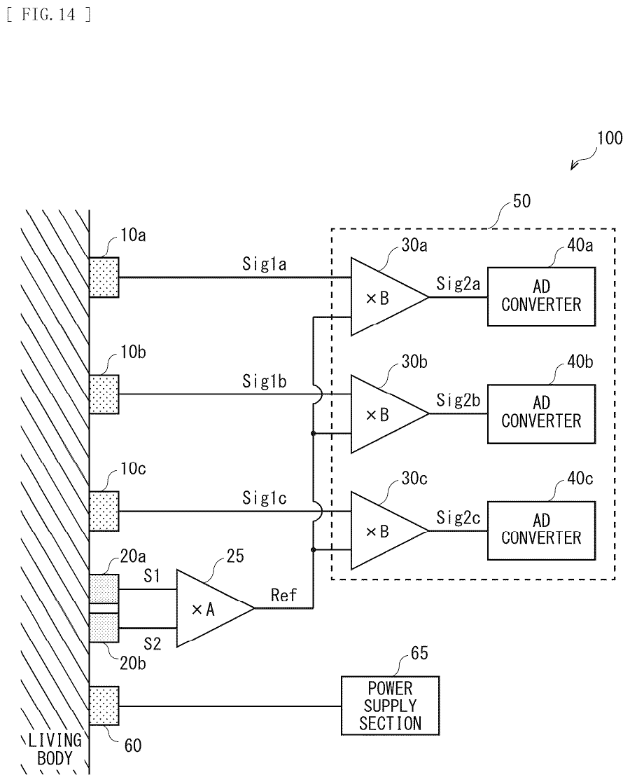

In addition, the reference electrodes 20a to 20c may each include a plurality of electrodes. As illustrated in FIG. 13, for example, the plurality of reference electrodes 20a may be disposed around the reference electrode 20b. In addition, a plurality of reference electrodes 20c may be disposed outside the plurality of reference electrodes 20a. As in the example illustrated in FIG. 13, a spacing between the reference electrode 20b in the middle and each of the reference electrodes 20a may be an equal spacing r. In addition, a distance between the reference electrode 20b in the middle and each of the reference electrodes 20c may be an equal spacing 2r.

### 2-2. Modification Example 2

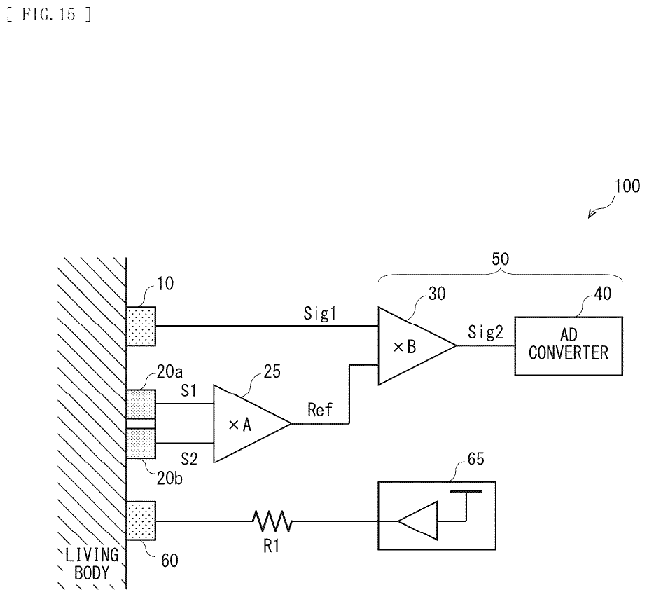

In the embodiment described above, the example is described in which the sensor unit 100 includes one measurement electrode, but the number and the arrangement of the measurement electrode are not limited to this. The sensor unit 100 may include two or more measurement electrodes. FIG. 14 is a diagram illustrating a configuration example of a sensor unit of a biosignal detection device according to Modification Example 2 of the present disclosure. In the present modification example, the sensor unit 100 of the biosignal detection device 1 includes measurement electrodes 10a to 10c. The measurement electrodes 10a to 10c are to be in contact with mutually different positions. As illustrated in FIG. 14, the AFE section 50 of the sensor unit 100 includes biosignal generators 30a to 30c and AD converters 40a to 40c.

The measurement electrode 10a is coupled to the biosignal generator 30a and supplies the biosignal generator 30a with a measurement signal Sigla corresponding to a potential of a site of a living body with which the measurement electrode 10a is in contact. The measurement electrode 10b is coupled to the biosignal generator 30b and supplies the biosignal generator 30b with a measurement signal Sig1b corresponding to a potential of a site of the living body with which the measurement electrode 10b is in contact. In addition, the measurement electrode 10c is coupled to the biosignal generator 30c and supplies the biosignal generator 30c with a measurement signal Sig1c corresponding to a potential of a site of the living body with which the measurement electrode 10c is in contact. The measurement signals Sigla to Sig1c are biosignals obtained from the measurement electrodes 10a to 10c, respectively.

In the present modification example, to each of a plurality of biosignal generators 30 (the biosignal generators 30a to 30c in FIG. 14), the reference signal generator 25 inputs the reference signal Ref. It becomes possible to use the reference signal Ref generated by the reference signal generator 25 as a reference signal common to the biosignal generators 30a to 30c.

The biosignal generator 30a outputs, to the AD converter 40a, a biosignal Sig2a based on a difference between a potential of the measurement signal Sigla and a potential of the reference signal Ref. The biosignal generator 30b outputs, to the AD converter 40b, a biosignal Sig2b based on a difference between a potential of the measurement signal Sig1b and the potential of the reference signal Ref. In addition, the biosignal generator 30c outputs, to the AD converter 40c, a biosignal Sig2c based on a difference between a potential of the measurement signal Sig1c and the potential of the reference signal Ref.

The AD converters 40a to 40c perform AD conversion processing on the biosignals Sig2a to Sig2c, respectively. The AD converter 40a performs the AD conversion of the biosignal Sig2a and outputs the biosignal Sig2a converted into a digital signal, to the signal processor 110 illustrated in FIG. 1. The AD converter 40b performs the AD conversion of the biosignal Sig2b and outputs the biosignal Sig2b converted into a digital signal, to the signal processor 110. In addition, the AD converter 40c performs the AD conversion of the biosignal Sig2c and outputs the biosignal Sig2c converted into a digital signal, to the signal processor 110.

In this manner, it is possible for the biosignal detection device 1 according to the present modification example to perform the detection of the plurality of biosignals Sig2a to Sig2c, using the plurality of measurement electrodes 10a to 10c. It becomes possible for the signal processor 110 and the estimation unit 120 to perform estimation of the state of the user, using the plurality of biosignals Sig2a to Sig2c.

### 2-3. Modification Example 3

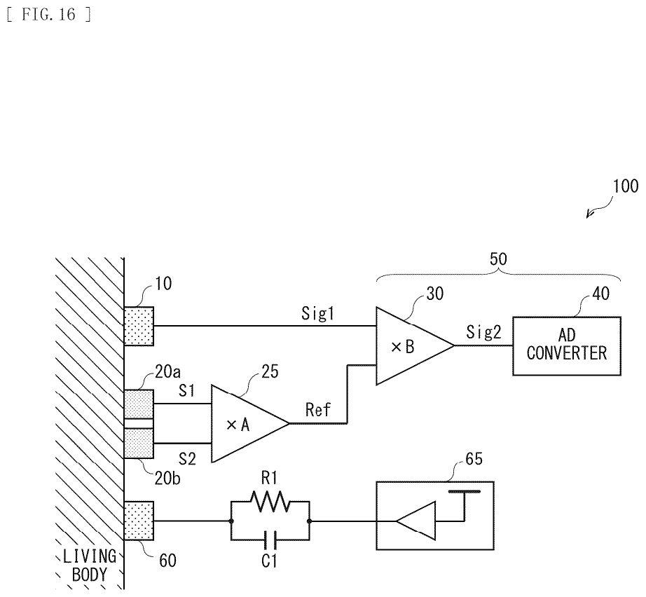

FIG. 15 is a diagram illustrating a configuration example of a sensor unit of a biosignal detection device according to Modification Example 3. The sensor unit 100 according to the present modification example includes a resistive element R1, as illustrated in FIG. 15. In addition, as schematically illustrated in FIG. 15, the power supply section 65 includes, for example, an amplifier circuit coupled to a power supply line. The power supply section 65 is a supply section that is configured to supply a voltage and is configured to supply a voltage to the bias electrode 60. The power supply section 65 may output a GND (ground) potential or a specific potential to the bias electrode 60.

The resistive element R1 is a resistor and provided between the power supply section 65 and the bias electrode 60. As illustrated in FIG. 15, the resistive element R1 is located between the power supply section 65 and the bias electrode 60, and is electrically coupled to the power supply section 65 and the bias electrode 60. The resistive element R1 is coupled in series between the power supply section 65 and the bias electrode 60.

One end of the resistive element R1 is coupled to the power supply section 65. Another end of the resistive element R1 is coupled to the bias electrode 60. In the example illustrated in FIG. 15, the bias electrode 60 is electrically coupled to the power supply section 65 via the resistive element R1, and a potential generated in the power supply section 65 is applied to the bias electrode 60. As a result, the reference signal generator 25 and the biosignal generator 30 that include the living body operate as a circuit. The reference signal generator 25 generates the reference signal Ref based on the difference between the potential of the signal S1 and the potential of the signal S2. In addition, the biosignal generator 30 may generate the biosignal Sig2 based on the difference between the potential of the measurement signal Sig1 and the potential of the reference signal Ref.

In the present modification example, the resistive element R1 is provided that is coupled in series between the power supply section 65 and the bias electrode 60. This reduces influence of the potential (the GND potential or the specific potential) of the power supply section 65 on the measurement signal Sig1 and the biosignals S1 and S2 inputted to the reference signal generator 25. It becomes possible to increase amplitude (signal level) of the biosignal Sig2 as compared with a case where the sensor unit 100 does not include the resistive element R1. Therefore, it becomes possible for the biosignal detection device 1 to detect the biosignal Sig2 having a larger amplitude than that in an existing measurement method even in a case where a distance between electrodes is short.

The biosignal detection device 1 according to the present modification example includes a fourth electrode (the bias electrode 60) that is configured to be in contact with the living body, a supply section (the power supply section 65) that is configured to supply a voltage, and a resistive element (the resistive element R1) coupled in series between the supply section and the fourth electrode. This makes it possible to detect the biosignal with high accuracy even in a case where the distance between the electrodes is small and to improve the detection performance of the biosignal. For example, it becomes possible to mount an electroencephalograph in a small earphone device such as a TWS (True Wireless Stereo) and acquire brain wave potentials larger than those in the existing measurement method.

### 2-4. Modification Example 4

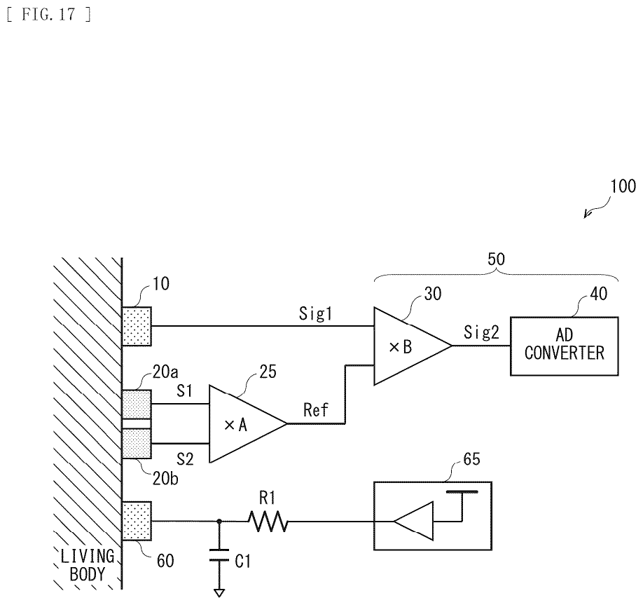

FIG. 16 is a diagram illustrating a configuration example of a sensor unit of a biosignal detection device according to Modification Example 4. In the example illustrated in FIG. 16, the sensor unit 100 includes a capacitive element C1, in addition to the resistive element R1. The capacitive element C1 is a capacitor (condenser) and is provided between the power supply section 65 and the bias electrode 60. As illustrated in FIG. 16, the capacitive element C1 is located between the power supply section 65 and the bias electrode 60, and is electrically coupled to the power supply section 65 and the bias electrode 60. The capacitive element C1 is coupled in series between the power supply section 65 and the bias electrode 60. The resistive element R1 and the capacitive element C1 are coupled in parallel.

One electrode (terminal) of the capacitive element C1 is coupled to the power supply section 65. Another electrode of the capacitive element C1 is coupled to the bias electrode 60. In the example illustrated in FIG. 16, the bias electrode 60 is electrically coupled to the power supply section 65 via the resistive element R1 and is also electrically coupled to the power supply section 65 via the capacitive element C1. A potential generated by the power supply section 65 is applied to the bias electrode 60. The reference signal generator 25 generates the reference signal Ref based on the difference between the potential of the signal S1 and the potential of the signal S2. In addition, the biosignal generator 30 may generate the biosignal Sig2 based on the difference between the potential of the measurement signal Sig1 and the potential of the reference signal Ref.

In the present modification example, provided are the resistive element R1 coupled in series between the power supply section 65 and the bias electrode 60, and the capacitive element C1 coupled in series between the power supply section 65 and the bias electrode 60. This reduces the influence of the potential of the power supply section 65 on the measurement signal Sig1 and the signals S1 and S2. Therefore, it becomes possible for the biosignal detection device 1 to detect the biosignal Sig2 having the larger amplitude than that in the existing measurement method even in a case where the distance between the electrodes is short.

The biosignal detection device 1 according to the present modification example includes the fourth electrode (the bias electrode 60) that is configured to be in contact with the living body, the supply section (the power supply section 65) that is configured to supply a voltage, the resistive element (the resistive element R1) coupled in series between the supply section and the fourth electrode, and a capacitive element (the capacitive element C1) coupled in series between the supply section and the fourth electrode. This makes it possible to detect the biosignal with high accuracy even in the case where the distance between the electrodes is small and to improve the detection performance of the biosignal. For example, it becomes possible to mount an electroencephalograph in a small earphone device such as the TWS and acquire the brain wave potential having a larger amplitude than that with the existing measurement method.

### 2-5. Modification Example 5

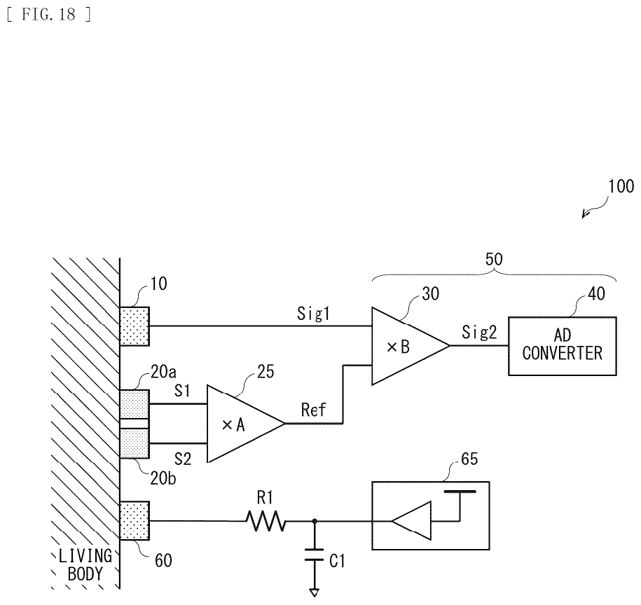

FIG. 17 is a diagram illustrating a configuration example of a sensor unit of a biosignal detection device according to Modification Example 5. As illustrated in FIG. 17, the sensor unit 100 includes the resistive element R1 and the capacitive element C1. The resistive element R1 is coupled in series between the power supply section 65 and the bias electrode 60. In the example illustrated in FIG. 17, the capacitive element C1 is provided between the bias electrode 60 and a grounding wire. The capacitive element C1 is located between the bias electrode 60 and the grounding wire, and is electrically coupled to the bias electrode 60 and the grounding wire. The one end of the capacitive element C1 is coupled to the bias electrode 60. The other electrode of the capacitive element C1 is coupled to the grounding wire.

In the sensor unit 100 of the biosignal detection device 1, the resistive element R1 and the capacitive element C1 being provided between the power supply section 65 and the bias electrode 60 reduces the influence of the potential of the power supply section 65 on the measurement signal Sig1 and the signals S1 and S2. It becomes possible to ensure the amplitude of the biosignal Sig2. Therefore, even in the case where the distance between the electrodes is short, it becomes possible to detect the biosignal Sig2 having the larger amplitude than that in the existing measurement method. In the case of the present modification example as well, it becomes possible to detect the biosignal with high accuracy and to improve the detection performance of the biosignal.

FIG. 18 is a diagram illustrating another configuration example of the sensor unit of the biosignal detection device according to Modification Example 5. As in the example illustrated in FIG. 18, the capacitive element C1 may be provided between the power supply section 65 and the grounding wire. In the example illustrated in FIG. 18, the capacitive element C1 is located between the power supply section 65 and the grounding wire, and is electrically coupled to the power supply section 65 and the grounding wire. The one electrode of the capacitive element C1 is coupled to the power supply section 65. The other electrode of the capacitive element C1 is coupled to the grounding wire. In this case as well, it becomes possible to reduce the influence of the potential of the power supply section 65 on the measurement signal Sig1 and the signals S1 and S2 and to ensure the amplitude of the biosignal Sig2.

It is to be noted that the arrangement of the resistive element R1 and the capacitive element C1 in the sensor unit 100 of the biosignal detection device 1 is not limited to the example described above. For example, the sensor unit 100 may include a plurality of resistive elements R1 and a plurality of capacitive elements C1.

### 2-6. Modification Example 6

It is possible to apply the technology according to the present disclosure to various products. The biosignal detection device 1 according to the present disclosure may be applied to, for example, wearable apparatuses such as earphone devices, headphone devices, or the like.

Although the present disclosure has been described above with reference to the embodiment and the modification examples, the present technology is not limited to the above-described embodiment and the like, and various modifications are possible. For example, the above-described modification examples have been described as the modification examples of the above-described embodiment, but it is possible to appropriately combine the configurations of the respective modification examples. In addition, the present disclosure has applicability not only to human bodies but also to living bodies other than the human bodies, such as animals including pet animals or farm animals.

The biosignal detection device according to one embodiment of the present disclosure includes the first electrode that is configured to be in contact with the living body; the second electrode and the third electrode that are configured to be in contact with the living body at respective positions different from that of the first electrode; and the first generator that generates the third signal related to the living body, on the basis of the first signal based on the potential of the first electrode and the second signal based on the potential of each of the second electrode and the third electrode. This makes it possible to perform the detection of the biosignal with high accuracy and to improve the detection performance of the biosignal.

It is to be noted that the effects described herein are merely illustrative and non-limiting, and other effects may be achieved. Moreover, the present disclosure may have the following configurations.

(1)

A biosignal detection device including:
a first electrode that is configured to be in contact with a living body;
a second electrode and a third electrode that are configured to be in contact with the living body at respective positions different from that of the first electrode; and
a first generator that generates a third signal related to the living body, on the basis of a first signal based on a potential of the first electrode and a second signal based on respective potentials of the second electrode and the third electrode.
   (2)

The biosignal detection device according to (1) described above, including
a second generator that generates the second signal based on a difference between the potential of the second electrode and the potential of the third electrode, in which
the first generator generates the third signal on the basis of the first signal and the second signal that is generated by the second generator.
   (3)

The biosignal detection device according to (2) described above, including
a comparator that compares the first signal and the second signal with each other, in which
the second generator outputs the second signal amplified by a gain that is set on the basis of a comparison result obtained by the comparator.
   (4)

The biosignal detection device according to any one of (1) to (3) described above, in which the first generator generates the third signal based on a difference between the first signal and the second signal that is a reference signal.

(5)

The biosignal detection device according to any one of (1) to (4) described above, including
a comparator that compares the first signal and the second signal with each other, in which
the first generator outputs the third signal amplified by a gain that is set on the basis of a comparison result obtained by the comparator.
   (6)

The biosignal detection device according to any one of (1) to (5) described above, including
a fourth electrode that is configured to be in contact with the living body, in which
the first generator generates the third signal based on a difference between the potential of the first signal and the potential of the second signal, using a potential of the fourth electrode as a reference potential.
   (7)

The biosignal detection device according to any one of (1) to (6) described above, including a signal processor that converts the third signal into intensity for each frequency.

(8)

The biosignal detection device according to (7) described above, in which the signal processor standardizes the third signal in a predetermined frequency range.

(9)

The biosignal detection device according to any one of (1) to (8) described above, in which the second electrode and the third electrode have respective concentric shapes.

(10)

The biosignal detection device according to any one of (1) to (9) described above, including:
a plurality of the third electrodes disposed around the second electrode; and
a second generator that generates the second signal based on a difference between the potential of the second electrode and potentials of the plurality of third electrodes.
   (11)

The biosignal detection device according to (10) described above, in which the plurality of third electrodes is disposed with equal spacings around the second electrode.

(12)

The biosignal detection device according to (2) or (3) described above, including
a plurality of the first electrodes, in which
the first generator is provided for each of the first electrodes, and
the second generator outputs the second signal to a plurality of the first generators.
   (13)

The biosignal detection device according to any one of (1) to (12) described above, including:
a fourth electrode that is configured to be in contact with the living body;
a supply section that is configured to supply a voltage; and
a resistive element that is coupled in series between the supply section and the fourth electrode.
   (14)

The biosignal detection device according to any one of (1) to (13) described above, including:
a fourth electrode that is configured to be in contact with the living body;
a supply section that is configured to supply a voltage;
a resistive element that is coupled in series between the supply section and the fourth electrode; and
a capacitive element that is coupled in series between the supply section and the fourth electrode.
   (15)

The biosignal detection device according to (14) described above, in which the resistive element and the capacitive element are coupled in parallel to each other.

(16)

The biosignal detection device according to any one of (1) to (15) described above, including:
a fourth electrode that is configured to be in contact with the living body;
a supply section that is configured to supply a voltage;
a resistive element that is coupled in series between the supply section and the fourth electrode; and
a capacitive element that is provided between the fourth electrode and a grounding wire.
   (17)

The biosignal detection device according to (16) described above, in which
a first electrode of the capacitive element is electrically coupled to the fourth electrode, and
a second electrode of the capacitive element is electrically coupled to the grounding wire.
   (18)

The biosignal detection device according to any one of (1) to (17) described above, including:
a fourth electrode that is configured to be in contact with the living body;
a supply section that is configured to supply a voltage;
a resistive element that is coupled in series between the supply section and the fourth electrode; and
a capacitive element that is provided between the supply section and the grounding wire.
   (19)

The biosignal detection device according to (18) described above, in which
a first electrode of the capacitive element is electrically coupled to the supply section, and
a second electrode of the capacitive element is electrically coupled to the grounding wire.
   (20)

The biosignal detection device according to any one of (13) to (19) described above, in which the first generator is configured to generate the third signal based on a difference between the potential of the first signal and the potential of the second signal, using a potential of the fourth electrode as a reference potential.

The present application claims the benefit of Japanese Priority Patent Application JP2021-198820 filed with the Japan Patent Office on Dec. 7, 2021 and Japanese Priority Patent Application JP2022-126216 filed with the Japan Patent Office on Aug. 8, 2022, the entire contents of which are incorporated herein by reference.

It should be understood by those skilled in the art that various modifications, combinations, sub-combinations and alterations may occur depending on design requirements and other factors insofar as they are within the scope of the appended claims or the equivalents thereof.

1. A biosignal detection device comprising:
a first electrode that is configured to be in contact with a living body;
a second electrode and a third electrode that are configured to be in contact with the living body at respective positions different from that of the first electrode; and
a first generator that generates a third signal related to the living body, on a basis of a first signal based on a potential of the first electrode and a second signal based on respective potentials of the second electrode and the third electrode.

2. The biosignal detection device according to claim 1, comprising
a second generator that generates the second signal based on a difference between the potential of the second electrode and the potential of the third electrode, wherein
the first generator generates the third signal on a basis of the first signal and the second signal that is generated by the second generator.

3. The biosignal detection device according to claim 2, comprising
a comparator that compares the first signal and the second signal with each other, wherein
the second generator outputs the second signal amplified by a gain that is set on a basis of a comparison result obtained by the comparator.

4. The biosignal detection device according to claim 1, wherein the first generator generates the third signal based on a difference between the first signal and the second signal that is a reference signal.

5. The biosignal detection device according to claim 1, comprising
a comparator that compares the first signal and the second signal with each other, wherein
the first generator outputs the third signal amplified by a gain that is set on a basis of a comparison result obtained by the comparator.

6. The biosignal detection device according to claim 1, comprising
a fourth electrode that is configured to be in contact with the living body, wherein
the first generator generates the third signal based on a difference between the potential of the first signal and the potential of the second signal, using a potential of the fourth electrode as a reference potential.

7. The biosignal detection device according to claim 1, comprising a signal processor that converts the third signal into intensity for each frequency.

8. The biosignal detection device according to claim 7, wherein the signal processor standardizes the third signal in a predetermined frequency range.

9. The biosignal detection device according to claim 1, wherein the second electrode and the third electrode have respective concentric shapes.

10. The biosignal detection device according to claim 1, comprising:
a plurality of the third electrodes disposed around the second electrode; and
a second generator that generates the second signal based on a difference between the potential of the second electrode and potentials of the plurality of third electrodes.

11. The biosignal detection device according to claim 10, wherein the plurality of third electrodes is disposed with equal spacings around the second electrode.

12. The biosignal detection device according to claim 2, comprising
a plurality of the first electrodes, wherein
the first generator is provided for each of the first electrodes, and
the second generator outputs the second signal to a plurality of the first generators.

13. The biosignal detection device according to claim 1, comprising:
a fourth electrode that is configured to be in contact with the living body;
a supply section that is configured to supply a voltage; and
a resistive element that is coupled in series between the supply section and the fourth electrode.

14. The biological detection device according to claim 1, comprising:
a fourth electrode that is configured to be in contact with the living body;
a supply section that is configured to supply a voltage;
a resistive element that is coupled in series between the supply section and the fourth electrode; and
a capacitive element that is coupled in series between the supply section and the fourth electrode.

15. The biosignal detection device according to claim 14, wherein the resistive element and the capacitive element are coupled in parallel to each other.

16. The biosignal detection device according to claim 1, comprising:
a fourth electrode that is configured to be in contact with the living body;
a supply section that is configured to supply a voltage;
a resistive element that is coupled in series between the supply section and the fourth electrode; and
a capacitive element that is provided between the fourth electrode and a grounding wire.

17. The biosignal detection device according to claim 16, wherein
a first electrode of the capacitive element is electrically coupled to the fourth electrode, and
a second electrode of the capacitive element is electrically coupled to the grounding wire.

18. The biological detection device according to claim 1, comprising:
a fourth electrode that is configured to be in contact with the living body;
a supply section that is configured to supply a voltage;
a resistive element that is coupled in series between the supply section and the fourth electrode; and
a capacitive element that is provided between the supply section and the grounding wire.

19. The biosignal detection device according to claim 18, wherein
a first electrode of the capacitive element is electrically coupled to the supply section, and
a second electrode of the capacitive element is electrically coupled to the grounding wire.

20. The biosignal detection device according to claim 13, wherein the first generator is configured to generate the third signal based on a difference between the potential of the first signal and the potential of the second signal, using a potential of the fourth electrode as a reference potential.
